(19) United States
(12) Patent Application Publication
(10) Pub. No.: US 2004/0111152 A1
(43) Pub. Date: Jun. 10, 2004

(54) Accommodating multifocal intraocular lens

(76) Inventor: Charles David Kelman, Boca Raton, FL (US)

Correspondence Address: DARBY & DARBY P.C., P. O. BOX 5257, NEW YORK, NY, 10150-5257, US

(21) Appl. No.: 10/316,132

(22) Filed: Dec. 10, 2002

(51) Int. Cl. A61F 2/16

(52) U.S. Cl. 623/6.37; 623/6.4

(57) ABSTRACT

An accommodating intraocular lens is provided and includes an optic; a haptic having an opening formed therein; and a flexible membrane having a first peripheral edge and a second peripheral edge. The flexible membrane has an opening extending therethrough from the first peripheral edge to the second peripheral edge, wherein the first peripheral edge attaches to the optic and the second peripheral edge attaches to the haptic so that the optic is axially aligned with the opening. The flexible membrane is formed of a memory type material so that when the ciliary muscle constricts, the optic moves in an anterior direction and the flexible membrane deforms from a first position to permit the optic to move. Relaxation of the ciliary muscle causes the optic to move in a posterior direction, resulting in both the optic and flexible membrane toward the first position.

### TECHNICAL FIELD

The present application generally relates to the field of intraocular lenses (IOLs) and more specifically, relates to intraocular lenses that have a plurality of optical powers and, in addition, are adapted to provide accommodating movement in the eye.

### BACKGROUND

The human eye includes an anterior chamber between the cornea and iris, posterior chamber, defined by a capsular bag, containing a crystalline lens, a vitreous chamber behind the lens containing the vitreous humor, and a retina at the rear of this chamber. Normally when a person focuses on an object disposed at a distance from the eye, focusing is achieved by virtue of the contraction of the ciliary muscles which affects the curvature of the lens and thereby its focal length. The process whereby the eye is able to focus on objects over a wide range of distances from the eye is called “accommodation”.

Natural accommodation in a normal human eye involves shaping of the natural crystalline lens by automatic contraction and relaxation of the ciliary muscle of the eye by the brain to focus the eye at different distances. Ciliary muscle relaxation shapes the natural lens for distant vision. Ciliary muscle contraction shapes the natural lens for near vision.

One common procedure during a cataract operation is to remove material from the lens capsule and replace it by an intraocular lens implant. The simplest of implants are fixed lenses having a single focal length. However, this type of lens has a number of disadvantages, namely that such a lens does not provide for any accommodation by the eye for the distance of objects and therefore, this type of lens has a relatively limited utility.

A more advanced type of IOL for implantation is one which provides a number of focal lengths. Some of the light impinging the lens is subjected to focusing at each of the different focal lengths of the lens. This type of lens does provide for a broader range of focus for the eye. Only a portion of the light; however, is focused on the retina of the eye for any of the focal lengths. Thus, if an object is focused by one of the focal lengths, only a certain percentage (typically less than 50%) of the light will be focused and the remaining percentage of the light will be only partly focused or unfocused. This causes the patient to experience a reduction of contrast of the focused object and a reduction in contrast sensitivity.

Despite advances in providing multi-focal IOLs for implantation in the eye, many of these lenses still suffer from the disadvantage that they typically do not provide accommodating movement. Attempts have been made to provide IOLs with accommodating movement along the optical axis of the eye as an alternative to shape changing. While these type of lenses made advances in the art, they also still suffer from a number of disadvantages. For example, one problem that exists with IOLs which are adapted for accommodating movement toward and away from the retina of the eye is that such IOLs cannot move sufficiently to obtain the desired accommodation because of space constraints within the eye and many of the proposed solutions are overly complex.

### SUMMARY

An accommodating intraocular lens (IOL) is provided and preferably has a plurality of optical powers and also provides enhanced accommodating movement within the eye.

In one embodiment, the accommodating intraocular lens includes an optic; a haptic having a central opening formed therein for receiving the optic; and a flexible membrane that is formed of a resilient biocompatible material and is designed as a means for coupling the optic to the haptic. The flexible membrane can be in the form of an annular member with a central opening extending therethrough from a first peripheral edge that attaches to the optic to a second peripheral edge that attaches to the haptic. The attachment between the optic and the flexible membrane is preferably such that the optic is axially aligned with the opening of the haptic. The biocompatible material that is used to form the flexible membrane has memory so that when the ciliary muscle constricts, an increase in vitreous pressure results and this causes the optic to move in an anterior direction. This results in the flexible membrane deforming from an initial relaxed state to permit the optic to move in the anterior direction. For example, one exemplary flexible membrane has one or more folds between the two peripheral edges and therefore, when the optic moves in the anterior direction, the flexible membrane expands by unfolding about its folds. Relaxation of the ciliary muscle causes a reduction in the vitreous pressure resulting in the flexible membrane moving back toward its initial state and the optic is drawn in a posterior direction.

The present IOL is uniquely constructed to utilize the same ciliary muscle action to provide for accommodation and more specifically, the present IOL is constructed so that it is responsive to pressure changes in the vitreous humor. The vitreous humor is contained in a vitreous cavity that is located behind the capsular bag that contains the implanted IOL in one exemplary application. When the ciliary muscle contracts and relaxes, there are corresponding changes in the vitreous pressure (i.e., contraction of the ciliary muscle increases the vitreous pressure and relaxation of the ciliary muscle decreases the vitreous pressure). The change in the vitreous pressure is the driving force that causes the anterior and posterior movement of the optic since the materials that are used to form the optic and flexible membrane and the arrangement of these members relative to the haptic is such that these pressure changes are directly translated to the optic and flexible member, thereby driving the two in either an anterior or posterior direction. Preferably, the optic is initially in a position where it is substantially coplanar with the haptic and the memory property of the flexible membrane returns the optic to this initial position. The present IOL thus provides for accommodation by utilizing a flexible membrane that is easy to construct and simple in design.

Further aspects and features of the present embodiments can be appreciated from the appended Figures and accompanying written description.

### BRIEF DESCRIPTION OF THE DRAWINGS

The foregoing and other features of the present embodiment will be more readily apparent from the following detailed description and drawings of illustrative embodiments in which:

FIG. 1A is a section through a normal eye illustrating implantation of an accommodating intraocular lens according to one exemplary embodiment;

FIG. 1B is a cross-sectional view of an accommodating intraocular lens according to one exemplary embodiment in a retracted position;

FIG. 2 is a cross-sectional view of the accommodating intraocular lens of FIG. 1 in a first extended position;

FIG. 3 is a cross-sectional view of the accommodating intraocular lens of FIG. 1 in a second extended position;

FIG. 4 is a cross-sectional view of an accommodating intraocular lens according to a second exemplary embodiment in a retracted position;

FIG. 5 is a cross-sectional view of the accommodating intraocular lens of FIG. 4 in an extended position;

FIG. 6 is a perspective view of an accommodating intraocular lens according to another embodiment;

FIG. 7 is a cross-sectional view of the accommodating intraocular lens of FIG. 6 illustrating movement of the optic;

FIG. 8 is a perspective view of an accommodating intraocular lens according to yet another embodiment; and

FIG. 9 is a cross-sectional view of the accommodating intraocular lens of FIG. 8 illustrating movement of the optic.

### DETAILED DESCRIPTION OF THE PREFERRED EMBODIMENT

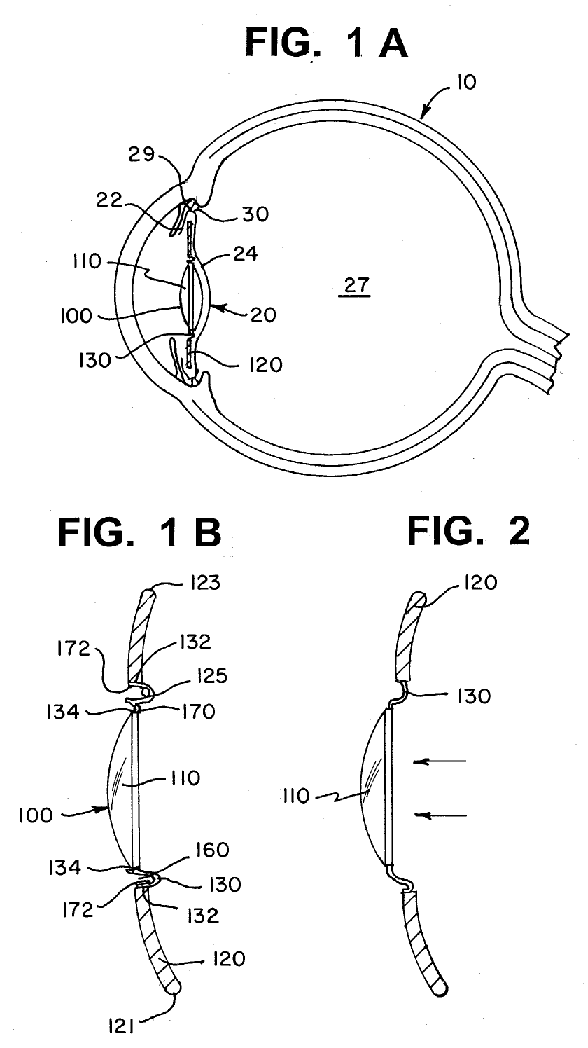

FIG. 1A is a cross-sectional view of a human eye 10 having an adaptive or accommodating IOL 100, in accordance with one exemplary embodiment, installed in place of the original material in a lens capsule 20. In FIG. 1, the cornea and other anterior portions of the eye are at the left of the figure and the retina and posterior portions of the eye are to the right. The IOL 100 includes an optic 110 sized and adapted for placement in a human eye, more specifically, the lens capsule (capsular bag) 20 thereof. The lens capsule 20 from which the original lens material has been removed, includes an annular anterior capsular remnant or rim 22 and an elastic posterior capsule (wall) 24 which are joined along the perimeter of the bag to form an annular crevice-like capsule bag sulcus 29 between the rim 22 and the posterior capsule 24. At least a portion of the original anterior rim 22 of the capsule 20 is generally removed during the operation for removal of the lens material leaving an opening 30, through which the IOL 100 is inserted. Typically, at least a portion of the posterior capsule 24 is left intact; however, as will be described hereinafter, a section of the posterior capsule 24 may be removed during the operation. The capsular rim 22 is the remnant of the anterior capsule that remains an operation (capsulorhexis) has been performed on the original lens. The capsular rim 22 circumferentially surrounds a central, generally round anterior opening in the capsular bag 20 though which the natural lens was previously removed. The capsular bag 20 is secured about its perimeter to the ciliary muscle of the eye by zonules 30. A vitreous cavity 27 is formed behind the capsular bag (lens capsule) 20 and is filled with the gel-like vitreous humor.

Referring to FIGS. 1A and 1B, the IOL 100 also includes a haptic (a plate haptic or a body including linkage arms or any other type of conventional haptic structure) 120 that serve to position and retain the IOL 100 in a desired location within the lens capsule 20. The haptic 120 can have any number of different shapes and sizes, as is know in the art, so long as the haptic 120 serves its intended purpose which is to locate and retain the IOL 100 within the lens capsule 20. In the illustrated embodiment, the haptic 120 is in the form of a simple plate haptic that is implanted within the eye 10 in a position where the lens optic 110 is aligned on the axis of the eye 10 with the anterior capsulotomy and outer ends 121, 123 of the plate haptic 110 are situated within the capsular bag sulcus 29 in contact with the sulcus wall. The normally posterior side of the lens then faces the elastic posterior capsule 24 of the bag 20.

The exemplary plate haptic 120 is generally rectangular in shape; however, the ends 121, 123 have arcuate shapes, is in known in the art, to provide comfort to the wearer of the IOL 100 when the IOL 100 is implanted into the eye 10. It will be understood that the haptic 110 is merely one example of a haptic that can be used in the present IOL 100. The plate haptic 120 comprises a body structure that surrounds the optic 110 and serves to not only locate but also support the optic 110 within the eye 10. More specifically, the plate haptic 120 has an opening 125 formed therein for receiving the optic 110. In the present embodiment, the opening 125 has a generally circular shape and the diameter thereof is greater than a diameter of the optic 110 to permit the optic 110 to be received completely within the opening 125 and to permit the optic 110 to freely move within the opening 125.

The IOL 100 also includes a biasing element 130 that is coupled between the plate haptic 120 and the optic 110 for assisting in providing the desired natural accommodation capability of the eye 10. The biasing element 130 is coupled to the haptic 120 at a first section 132 thereof and is coupled to the optic 110 at a second section 134 thereof in such a manner that when a force is applied to or removed from a posterior side of the IOL 100, the optic 110 will move accordingly. More specifically, the biasing element 130 thus permits the optic 110 to move anteriorly and posteriorly within the eye 10 when a respective force is applied or removed from the posterior side of the optic 110.

According to one embodiment, the biasing element 130 is formed of a material having an elastic memory and in an unstressed configuration, the haptic 120, optic 110, and the biasing element 130 are disposed substantially in a common plane. In a stressed condition, when a force is applied to the posterior side of the IOL 100, the biasing element 130 deforms and stores energy, resulting in the optic 110 traveling along the axis of the eye. For example, the biasing element 130 can be in the form of a flexible membrane with the first section 132 thereof being one edge of the membrane 130 and the second section 134 is another edge of the membrane 130. The flexible membrane 130 is constructed of a flexible material having memory and is constructed so that it can readily store energy by increasing its relative dimensions, as for example by unfolding when pressure is applied. In this embodiment, the flexible membrane 130 includes one or more folds so that in a relaxed condition, the flexible membrane 130 is folded into a relatively compact structure and when pressure is applied to the flexible membrane 130, it unfolds along the fold lines, thereby increasing its relative dimensions.

The flexible membrane 130 is formed of one of the suitable biocompatible materials discussed below or any other type of suitable biocompatible material that has sufficient memory characteristics. Preferably, the flexible membrane 130 is formed of a material that is more flexible than the material that is used to form both the optic 110 and the haptic 120. The flexible membrane 130 and the haptic 120 can be formed of a transparent material; however, this is not a requirement and it will be appreciated that these elements can be semi-transparent or even opaque.

As previously mentioned, the optic 110 has a diameter less than a diameter of the opening 125 of the haptic 120 so that the optic 110 is free to sit within the opening 125 and is also free to move out of and return back to a position of rest where it is disposed at least partially within the opening 125. Because of these relative dimensions, a gap 160 is formed between a peripheral edge 170 of the optic 110 and a peripheral edge 172 of the haptic 120 that defines the opening 125 formed therein.

The first section 132 of the flexible membrane 130 is attached to the plate haptic 120 at the peripheral edge 172 thereof and the second section 134 of the flexible membrane 130 is attached to the optic 110 at the peripheral edge 170 thereof. One exemplary flexible membrane 130 is thus a flexible ring-link structure having an annular opening formed therein and defined by a periphery of the second section 134 for receiving the optic 110 and the first section 132 represents an annular outer peripheral edge of the flexible membrane 130.

The IOL 100 can be and is preferably formed as a unitary member using traditional techniques, such as a molding process. While the IOL 100 is formed as an integral, single member according to one exemplary embodiment, the IOL 100 can also be formed as an assembly of two or more components. For example, the haptic 120 and the flexible membrane 130 can be formed as one component and then the optic 110 can be formed as a second component and attached to the second section 134 of the flexible membrane 130 using any number of conventional techniques, including a bonding process (e.g., heat bonding) or using an adhesive or the like, or any other type of process that results in the second section 134 of the flexible membrane 130 being attached to the optic 110. Because the optic 110 and the opening 125 have complementary shapes (e.g., circular), the flexible membrane 130 in its relaxed condition has a shape that is generally complementary to these other members and is designed to sit within and seal the gap 160.

FIGS. 1A and 1B show the relative positions of the optic 110, haptic 120 and the flexible membrane 130 in a relaxed position with the flexible membrane having released its energy and the optic 110 being substantially coplanar with the haptic 120. In this relaxed position, the flexible membrane 130 is folded along one or more fold lines and therefore, is in a relatively compact state in the gap 160 between the optic 110 and the haptic 120. Because, the flexible membrane 130 is in its relaxed state/position, it is free of storing any energy and therefore, the optic 110 is held in a position where it is at least partially drawn into the opening 125 of the haptic 120 so that it is substantially coplanar with the haptic 120.

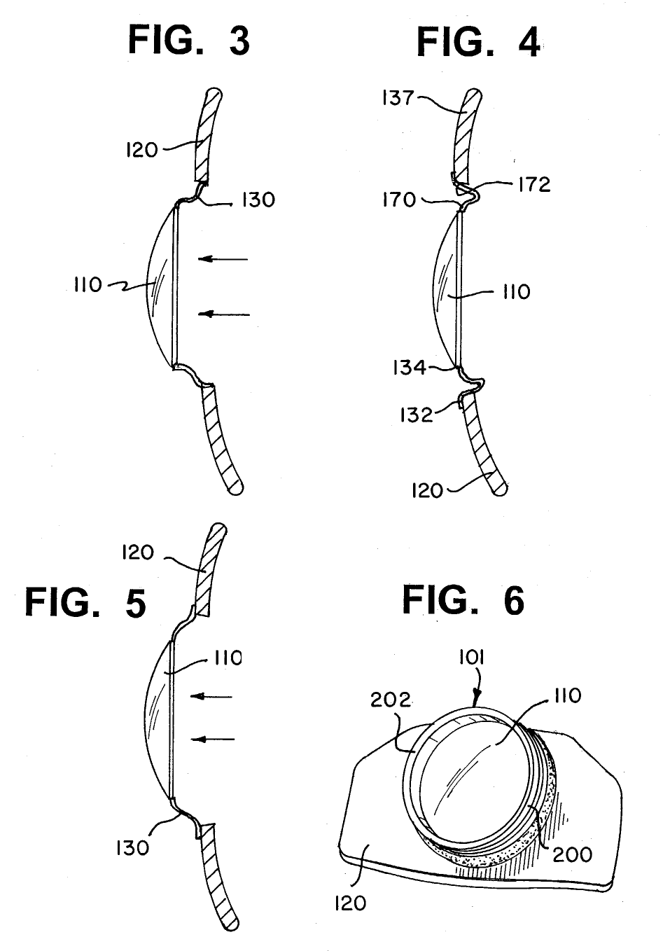

FIGS. 2 and 3 show the relative positions of the optic 110, haptic 120 and the flexible membrane 130 after an initial force has been applied to the posterior side of the optic 110. Such a force applied to the posterior side of the IOL 100 can be the result of a build-up in the vitreous pressure within the vitreous cavity 27 as a result of normal eye movement, such as when the ciliary muscle of the eye contracts to accommodate for near vision. This movement results in the equatorial diameter of the lens capsule 20 changing and a force is exerted on the outer ends 121, 123 of the haptic 120. Because the haptic 120 is preferably formed of a biocompatible material that is more rigid than both the optic 110 and the flexible membrane 130, the haptic 120 preferably does not move, or only very slightly, in response to the ciliary muscle either expanding or contracting. Instead, the flexible membrane 130 is the member that is designed to respond to normal eye movements in a manner that provides for accommodation. It will be appreciated that in order for the vitreous fluid to act as the biasing force that applies pressure to the flexible membrane 130 to cause an expansion thereof (unfolding or elongation action), the elastic posterior capsule 24 can be modified by forming at least one or more openings therein to permit the vitreous humor to contact or sufficiently apply a force (pressure) to the optic 110 and the flexible membrane 130 so as to facilitate movement of the optic 110 in an anterior direction away from the plate haptic 120. Preferably, an opening formed in the elastic posterior capsule 24 is aligned with the optic 110 to permit the vitreous humor to apply the above described force to the optic 110.

More specifically, when the ciliary muscle of the eye is relaxed, for distance vision, the haptic 120 and the flexible membrane 130 are in the relaxed position, the optic 110 is held in one position which focuses distant images onto the retina. When the ciliary muscle of the eye contracts to accommodate for near vision, the equatorial diameter of the lens capsule 20 changes, exerting force on the outer ends 121, 123 of the haptic 120 causing the haptic 120 to move slightly; however, at the same time, a pressure build-up of the vitreous fluid also results. The vitreous fluid applies a force against the posterior side of the IOL 100, including the optic 110, and because the optic 110 is not rigidly attached to the haptic 110 but is permitted a degree of movement relative thereto due to the flexible membrane 130 which couples the optic 110 to the haptic 120. In other words, the vitreous fluid applies pressure against the optic 110, as well as the flexible membrane 130, and this causes the flexible membrane 130 to store energy and deform by becoming unfolded as the optic 110 is shifted forward, away from the retina, so as to focus near images onto the retina. When the ciliary muscle again relaxes, the vitreous pressure build-up lessens and the flexible membrane 130 begins to return to its relaxed state by releasing stored energy due to it being formed of a memory type material. As the flexible membrane 130 returns to its relaxed condition, it draws the optic 110 rearward toward the retina so as to return the optic 110 to its previous position of distant focus.

It will be appreciated that the first and second sections 132, 134 of the flexible membrane 130 can attach to their respective members at any number of different points. For example, the first section 132 that is coupled to the haptic 120 can be attached thereto along a peripheral edge 172 of the haptic 120 that defines the opening 125. Alternatively, the first section 132 can attach to either a top face 137 of the haptic 120 (as shown in FIGS. 4-5) or it can be attached to a bottom face of the haptic 120. Similarly, the second section 134 can be attached to a number of different points of the optic 110. Preferably, the second section 134 attaches to a peripheral side edge 170 of the optic 110; however, it can be attached to the optic 110 at other locations thereof.

FIGS. 4 and 5 illustrate an exemplary IOL 100 that is identical to the one disclosed in FIGS. 1-3 with the exception that the first section 132 of the flexible membrane 130 is attached to the top face 137. As with the prior embodiment, the flexible membrane is folded or is otherwise disposed in a compact state within the gap 160 between the optic 110 and the plate haptic 120. When the vitreous pressure builds up due to normal eye movement, a force is exerted on the posterior side of the optic 110 and this causes the optic 110 to move in an anterior direction away from the retina. The material characteristics of the flexible membrane 120 permit the optic 110 to move in this manner since the flexible membrane 120 can readily deform, such as by unfolding, etc., to permit movement of the optic 110. When the vitreous pressure abates as a result of normal eye movements, the flexible membrane 130 releases its stored energy and returns to its relaxed condition due to it being formed of a memory type elastic material.

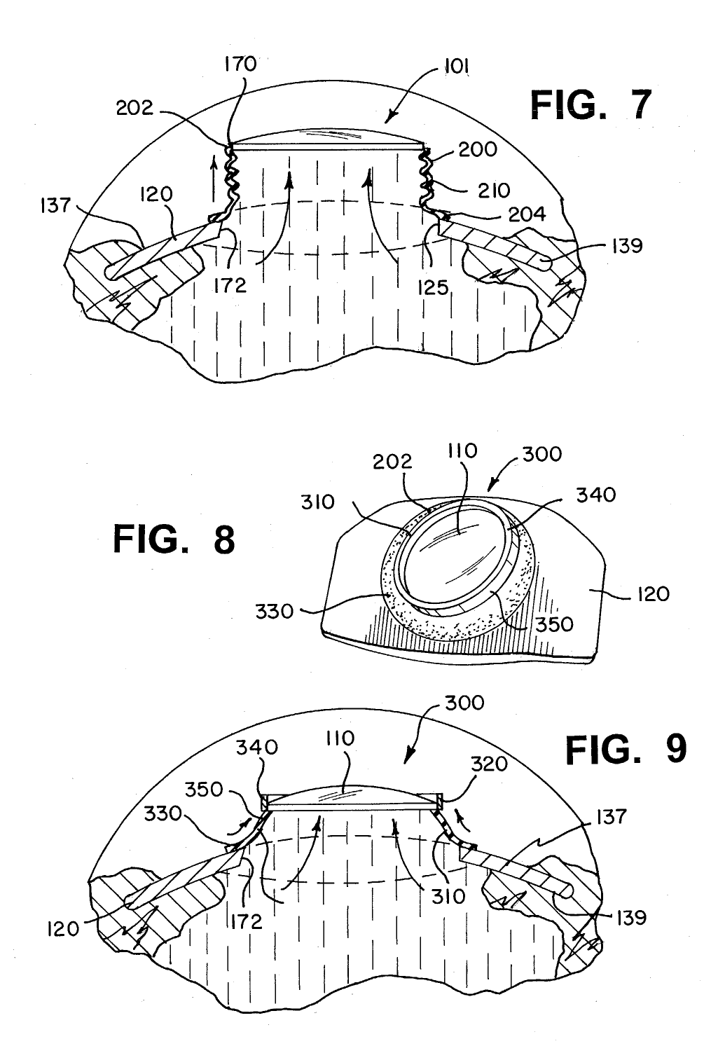

In another embodiment illustrated in FIGS. 6-7, an accommodating intraocular lens 101 according to another embodiment is provided with the flexible membrane being in the form of a bellows 200 that has an upper face 202 and an opposing lower face 204. As is known, a bellows is a member that expands and contracts due to it having accordion-like walls. The bellows 200 has a folded wall (accordion-like) 210 and a central opening 212 through which the axis of the eye passes therethrough. In one exemplary embodiment, the bellows 200 has an annular shape and the central opening 212 receives the optic 110.

The bellows 200 is formed of one of the suitable biocompatible materials discussed below or any other type of suitable biocompatible material. Preferably, the bellows 200 is formed of a material that is more flexible than the material that is used to form both the optic 110 and the haptic 120. The bellows 200 and the haptic 120 can be formed of a transparent material; however, this is not a requirement and it will be appreciated that these elements can be semi-transparent or even opaque.

The bellows 200 is disposed between the optic 110 and the plate haptic 120 by coupling the lower face 204 thereof to the plate haptic and coupling the upper face 202 to the optic 110. The material which is used to make the flexible membrane 130 (FIG. 1) can be used to make the bellows 200. The coupling of the upper and lower faces 202, 204, respectively, to the corresponding structures can be accomplished by conventional methods, including using an adhesive material, heat bonding the respective members, or otherwise causing the respective structures to bond or otherwise become securely attached to one another. Moreover, it will be appreciated that the IOL 101 can be formed as an integral member in which the haptic 120, the bellows 200 and the optic 110 are made as a single integral unit. Molding methods are available where an integral member can be formed to include different section that are formed of different materials, to thus give different material characteristics to the different sections.

As with the first embodiment, the optic 110 is preferably sized so that it can travel into and out of the opening 125 formed in the haptic 120 and so that it is centrally located therein. In other words, the diameter of the optic 110 is less than the diameter of the opening 125. Preferably, the optic 110 is substantially planar with the plate haptic 120 when the bellows 200 is in its relaxed position. The lower face 202 can therefore be attached to any number of different locations of the plate haptic 120. For example, the lower face 204 can be attached to the peripheral edge 172 of the haptic 120 or the lower face 204 can be attached to the top face 137 of the haptic 120 or it can be attached to a bottom face 139 of the haptic 120. The upper face 202 is preferably attached to the peripheral edge 170 of the optic 110.

Similar to the other embodiments, when the ciliary muscle of the eye is relaxed, for distance vision, the haptic 120 and the bellows 200 are in the relaxed position, the optic 110 is held in one position (preferably substantially coplanar with the haptic 120) which focuses distant images onto the retina. When the ciliary muscle of the eye contracts to accommodate for near vision, the equatorial diameter of the lens capsule 20 changes, exerting force on the outer ends of the haptic 120 causing the haptic 120 to move slightly; however, at the same time, a pressure build-up of the vitreous fluid also results. The vitreous fluid applies a force against the posterior side of the IOL 101, including the optic 110, and because the optic 110 is not rigidly attached to the haptic 120 but is permitted a degree of movement relative thereto due to the action of the bellows 200 which can store and release energy when pressure is applied and relieved, respectively, the optic 110 moves accordingly.

When the vitreous fluid applies pressure against the optic 110, as well as the bellows 200, the accordion-like wall 210 of the bellows 200 begins to deform by lengthening (i.e., unfold) as a result of the bellows 200 storing energy due to the applied force. As the bellows 200 becomes extended, the optic 110 is shifted forward, away from the retina, so as to focus near images onto the retina. When the ciliary muscle again relaxes, the vitreous pressure build-up lessens and the bellows 200 begins to return to its relaxed state by releasing stored energy due to it being formed of a memory type material. As the bellows 200 returns to its relaxed condition by contracting, it draws the optic 110 rearward toward the retina so as to return the optic 110 to its previous position of distant focus.

The different elastic characteristics of the optic 110, haptic 120 and the flexible membrane 130/bellows 200 can be provided by either using different materials, having different elastic properties, to form each component or the same material can be used to form all the components with the thickness of each component being controlled to either provide more or less elasticity. If it is desired to form the IOL as a unitary member using a molding process, then either different materials can be used in the process to form the individual components of different elasticity or the mold dies can be fabricated so that the individual components are formed of different thicknesses, thereby creating components having different elasticities.

Now referring to FIGS. 8-9 in which an accommodating intraocular lens 300 according to another embodiment is shown. In this embodiment, the biasing element is in the form of a flexible membrane 310 having a flange-like structure 320 for holding the optic 110 in place. More specifically, the flexible membrane is similar to the earlier described biasing elements and includes a bottom face that is in the form of a first flange 330 that serves as the attachment point between the flexible membrane 310 and the haptic 120. The flange 330 is attached to the haptic 120 or preferably is made as an integral part thereof so that the optic 110 is centered within the opening 125. The flange 330 can be attached to the haptic 120 in a number of different locations, such as the peripheral edge 172 of the haptic 120, the top face 137, or the bottom face 139.

The flexible membrane 310 also includes a second flange 340 with a resilient body section 350 formed between the first and second flanges 330, 340 (preferably, the flexible membrane 310 is formed of one material). In this embodiment, the optic 110 is nested or cradled underneath the second flange 340. More specifically, the optic 110 is nested between the second flange 340 and an annular fold section of the body section 350. In other words, the optic 110 is nested and held between two annular sections of material. The accommodating action of the IOL 300 is the same as in the other embodiments as in the change in the vitreous pressure is the means that precipitates the movement of the optic 110.

The optic 110 produces an image on the retina at the back of the eye 10 corresponding to a focal plane. In order to provide accommodation, the optic 110 according to the illustrated exemplary embodiment is made capable of movement along optical axis 40. As in the normal eye, accommodation is made consequent to changes in tension of the zonular fibers. This change in tension acts on the optic 110 so as to alter the image distance from the optic 110 to the focal plane.

The optic 110 can be formed of relatively hard material (rigid) biocompatible materials, relatively soft flexible semi-rigid material, or a combination of both hard and soft materials. Examples of relatively hard materials which are suitable for the optic 110 are polymethyl methacrylate (PMMA), polysulfones, and other relatively hard biological inert optical materials. Examples of suitable relatively soft materials for the optic 110 are silicone polymeric material, acrylic polymeric material, hydrogel polymeric material, thermolabile materials and other flexible semi-rigid biologically inert optical materials that enable the optic 110 to be rolled or folded for insertion through a small incision into the eye. It will also be appreciated that the IOL 100 can include either a refractive lens body according to one embodiment or a diffractive lens body according to another embodiment.

The optic 110 represents a central optical zone of the IOL. The optic 110 can be of a multi-focal type having different regions of differing optical power. For example, the optic 110 can include a central zone and a number of other radially zones spaced therefrom (e.g., inner and outer near zones and annular far zones). According to one embodiment, the central zone is circular and the peripheries of the annular zones are circular. The annular zones circumscribe the central zone and the zones are contiguous. These annular zones can be concentric and coaxial with the optic 110. The number and specifics of these zones will vary depending upon the precise lens application since these zones define vision corrective properties of the IOL.

The present IOLs are typically implanted within the capsular bag within the eye while the ciliary muscle is paralyzed in its relaxed state and the capsular bag is thereby stretched to its maximum diameter. The overall length of the IOL, as measured between the ends of the haptics 120, when the haptics are in their normal unstressed condition or state is slightly greater than the inner diameter of the capsule bag. However, other techniques can be used to implant the IOLs.

It will also be appreciated that the implantation of the present IOLs in the capsular bag 20 is merely only one application since it is well know that IOLs can be implanted in a number of other locations besides the capsular bag 20.

The foregoing written description is of a preferred embodiment and particular features of the present invention and is not restrictive of the many applications or the breadth of the present invention which is instead defined by the claims appended hereto and substantial equivalents thereof.

What is claimed is:

1. An accommodating intraocular lens comprising:
an optic;
a haptic having an opening formed therein;
a flexible membrane having a first peripheral edge and a second peripheral edge, the flexible membrane having an opening extending therethrough from the first peripheral edge to the second peripheral edge, wherein the first peripheral edge attaches to the optic and the second peripheral edge attaches to the haptic so that the optic is axially aligned with the opening, the flexible membrane being formed of a memory type material so that when a ciliary muscle constricts, the optic moves in an anterior direction and the flexible membrane deforms from a first position to permit the optic to move and wherein relaxation of the ciliary muscle causes the optic to move in a posterior direction, resulting in both the optic and flexible membrane moving toward the first position.

2. The accommodating intraocular lens of claim 1, wherein the haptic comprises a plate haptic that completely surrounds the optic.

3. The accommodating intraocular lens of claim 1, wherein the flexible membrane comprises a thin layer of biocompatible material that has one or more folds between the first and second peripheral edges in the first position.

4. The accommodating intraocular lens of claim 1, wherein the flexible membrane is formed of a material having greater elasticity than a material of the haptic.

5. The accommodating intraocular lens of claim 1, wherein the flexible membrane and optic are formed of materials that are susceptible to movement in anterior and posterior directions due to a pressure increase and decrease in the vitreous cavity.

6. The accommodating intraocular lens of claim 1, wherein a length of the flexible membrane increases as the flexible membrane moves in the anterior direction from its first position.

7. The accommodating intraocular lens of claim 1, wherein the optic, flexible membrane and haptic are formed as an integral molded unit.

8. The accommodating intraocular lens of claim 1, wherein the first peripheral edge attaches to an annular peripheral edge of the optic.

9. The accommodating intraocular lens of claim 1, wherein the second peripheral edge attaches to an annular peripheral edge of the haptic that defines the opening.

10. The accommodating intraocular lens of claim 1, wherein the second peripheral edge attaches to one of a top face and a bottom face of the haptic which is in the form of a plate haptic.

11. The accommodating intraocular lens of claim 1, wherein the optic is substantially coplanar with the haptic when the flexible membrane is in the first position.

12. An accommodating intraocular lens comprising:
an optic;
a haptic having an opening formed therein;
a bellows formed of a resilient biocompatible material and including an upper face and an opposing lower face, the bellows having an central opening extending therethrough from the upper face to the lower face, wherein the upper face attaches to the optic and the lower face attaches to the haptic so that the optic is axially aligned with the bellows opening, the biocompatible material having a memory property so that when ciliary muscles constrict and an increase in vitreous pressure results, the optic moves in an anterior direction and the bellows deforms from a first position to permit the optic to move in the anterior direction and wherein relaxation of the ciliary muscles causes a reduction in the vitreous pressure resulting in the bellows moving back toward its first position and the optic being drawn in a posterior direction.

13. The accommodating intraocular lens of claim 12, wherein the haptic comprises a plate haptic that completely surrounds the optic.

14. The accommodating intraocular lens of claim 12, wherein the bellows comprises an annular member that has an accordion-shaped wall between the upper and lower faces, the accordion-shaped wall being capable of unfolding when the vitreous pressure increases and retracting when the vitreous pressure decreases.

15. The accommodating intraocular lens of claim 12, wherein the bellows is formed of a material having greater elasticity than a material that forms the haptic, the optic being formed a material that has an elasticity between the elasticity of the bellows and the haptic.

16. The accommodating intraocular lens of claim 12, wherein the bellows and optic are formed of materials that are susceptible to movement in anterior and posterior directions due to the pressure increase and decrease of vitreous humor in a vitreous cavity.

17. The accommodating intraocular lens of claim 12, wherein the optic, flexible membrane and haptic are formed as an integral molded unit.

18. The accommodating intraocular lens of claim 12, wherein the upper face attaches to an annular peripheral edge of the optic.

19. The accommodating intraocular lens of claim 12, wherein the lower edge attaches to an annular peripheral edge of the haptic that defines the opening.

20. The accommodating intraocular lens of claim 12, wherein the lower face attaches to one of a top face and a bottom face of the haptic that is in the form of a plate haptic.
